(19) United States
(12) Patent Application Publication
(10) Pub. No.: US 2022/0331464 A1
(43) Pub. Date: Oct. 20, 2022

(54) CLAMP ARM TYPE DISINFECTION DEVICE

(71) Applicant: OXTI CORPORATION, New Taipei City (TW)

(72) Inventor: Chih-Feng Ho, New Taipei City (TW)

(21) Appl. No.: 17/232,159

(22) Filed: Apr. 16, 2021

(51) Int. Cl. A61L 2/10

(52) U.S. Cl. CPC A61L 2/10; A61L 2202/11

(57) ABSTRACT

A clamping arm type disinfection device includes: a main body; a disinfection lamp, configured on the main body, and including a light-emitting unit, sensing unit, control unit, and power unit; a clamping part, configured on one side of the main body, and including two clamping claws configured oppositely to each other, at least one of the two clamping claws configured with a pivot shaft, the two clamping claws abutting against each other to form a clamping space, and the two clamping claws respectively configured with a through hole; a fixing part, passed through the through holes of the two clamping claws; and a notice board, configured on one side of the clamping part, and configured with an accommodating hole. in use, the main body is put around the door handle, and the clamping claws are clamped on the door handle; the disinfection lamp emits light to illuminate the door handle.

### TECHNICAL FIELD OF THE INVENTION

The present invention relates to a disinfection device, and more particularly to a clamp arm type disinfection device.

### DESCRIPTION OF THE PRIOR ART

In the past, infectious diseases have changed the human history many times, and infectious diseases are one of the causes of most deaths in the world. With the change of human life style, the global climate change changes the transmission route and distribution area of infectious diseases; viruses and pathogenic microorganisms continue to adapt and mutate, causing every kind of emerging infectious diseases and re-emerging infectious diseases to happen incessantly; from early measles, smallpox, malaria, influenza to the recent severe acute respiratory syndrome (SARS), new type A influenza virus (H1N1), Middle East respiratory syndrome (MERS-CoV) and new coronavirus pneumonia (COVID-19), the currently known infectious transmission of such infectious diseases is mainly spread by direct contact with viral secretions or droplets.

Patients cause viruses to attach to doorknobs or door handles due to coughing, sneezing or contact with respiratory secretions, such that the doorknobs or door handles are highly contagious routes of the viruses. Chinese Patent NO. CN211818657U, titled “Door handle squeeze sterilizer”, discloses a disinfection device including a handle glove and a liquid chamber configured in the handle glove, where the handle glove is configured with an insertion hole for a door handle, with which the handle glove can be put around a door handle. When someone closes or opens a door with their hand, the pull force acted on the door handle by the hand will press the liquid chamber, and the disinfectant inside the liquid will thus be squeezed onto the hand.

However, the disinfectant is corrosive, irritating to skin, and has a bleaching effect and irritating odor harmful to human. In addition, the volume of the disinfectant determines the disinfection times, and as the number of times increases, it also means that the overall volume increases.

### SUMMARY OF THE INVENTION

To overcome the above disadvantages, the present invention proposes a clamping ann type disinfection device, including: a main body; a disinfection lamp, configured on the main body, and including a light-emitting unit, sensing unit, control unit, and power unit; a clamping part, configured on one side of the main body, and including two clamping claws configured oppositely to each other, at least one of the two clamping claws configured with a pivot shaft, the two clamping claws abutting against each other to form a clamping space, and the two clamping claws respectively configured with a through hole; a fixing part, passed through the through holes of the two clamping claws; and a notice board, configured on one side of the clamping part, and configured with an accommodating hole.

Preferably, the two clamping claws are respectively configured with a recess, allowing the two clamping claws to abut against each other to form the clamping space when closed.

Preferably, the main body is configured with a power receiving slot.

Preferably, the disinfection lamp includes a receiving unit, and the receiving unit is a wireless signal receiver.

Preferably, the disinfection lamp includes a receiving unit, and the receiving unit is a wireless signal receiver.

Taking a door handle as an example, the main body is put around the door handle through the accommodating hole of the notice board, and the clamping claws are then used to clamp the door handle; thus, the disinfection lamp emits light to illuminate the door handle for disinfection. Furthermore, the sensing unit is used to detect around the door handle. When the someone is sensed approaching, the control unit controls the disinfection lamp to stop emitting light. In addition, the main body is further configured with the power receiving slot to receive the power unit, and the disinfection lamp further includes the receiving unit to receive a wireless signal, allowing the control unit to control the light-emitting unit to emit light according to the wireless signal. Furthermore, the notice board may be provided with signs such as trademarks, product descriptions, use of warnings or decorative patterns.

### BRIEF DESCRIPTION OF THE DRAWINGS

FIG. 1 is a schematically perspective view of a clamping arm type disinfection device of the present invention;

FIG. 2 is a schematically perspective view of the present invention in a state of a clamping part being opened;

FIG. 3 shows a power receiving slot;

FIG. 4 is a block diagram of a disinfection lamp;

FIG. 5 shows an accommodating hole of a notice board put around a door handle;

FIG. 6 shows clamping claws in a state of clamping the door handle;

FIG. 7 shows a fixing part passed through through holes of the clamping claws;

FIG. 8 shows the disinfection lamp in a state of emitting light to illuminate the door handle; and

FIG. 9 shows the disinfection lamp in a state of stopping emitting light when someone approaches.

### DETAILED DESCRIPTION OF THE PREFERRED EMBODIMENTS

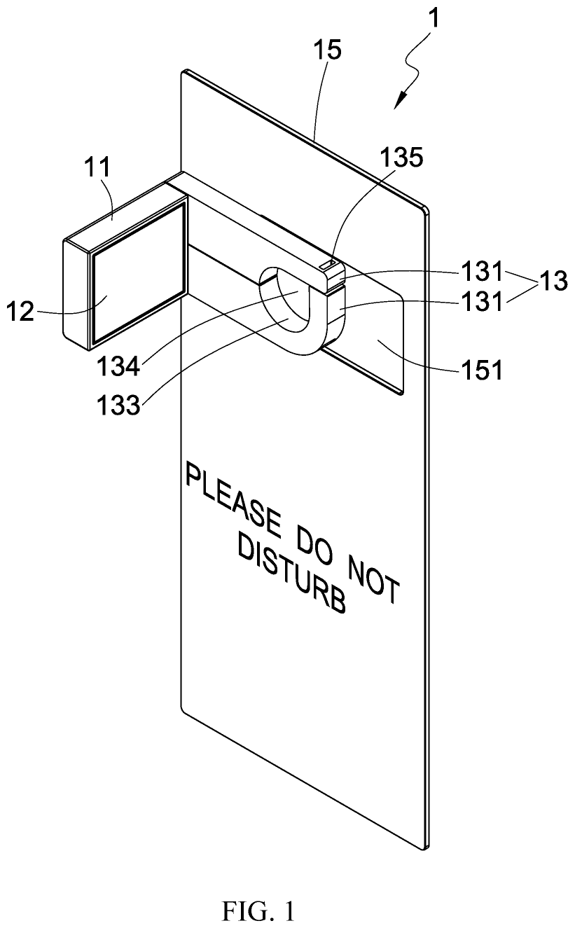

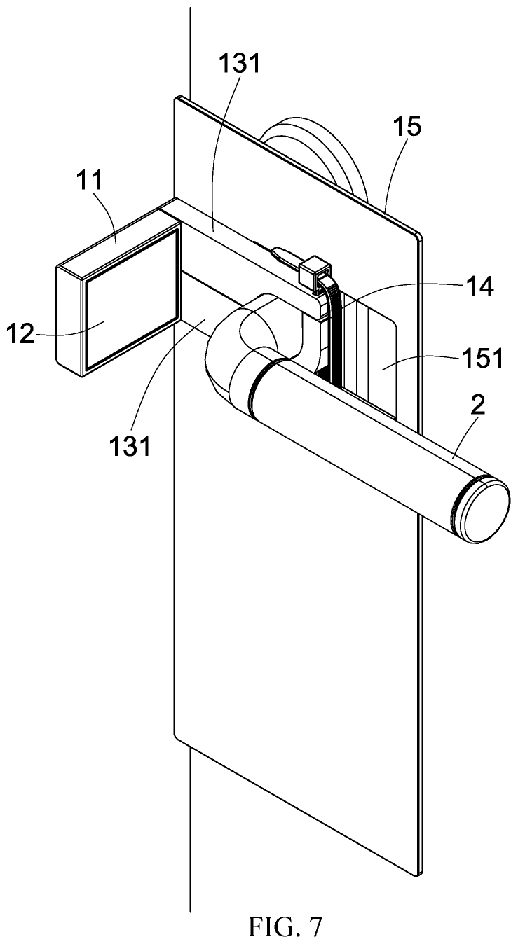

Referring to FIG. 1, a clamp arm type disinfection device includes a main body 11, disinfection lamp 12, clamping part 13, fixing part 14 (shown in FIG. 7), and notice board 15.

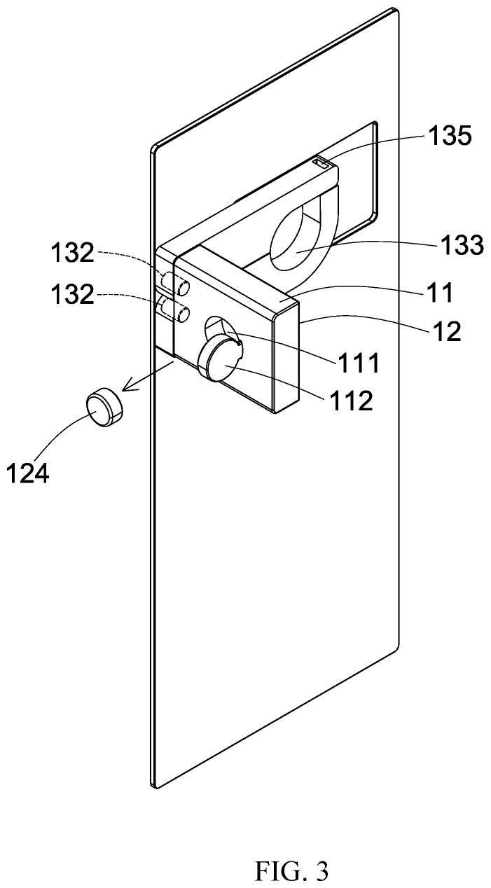

Referring to FIG. 3, the main body 11 may be configured with a power receiving slot 111, on which a protective cover 112 is configured.

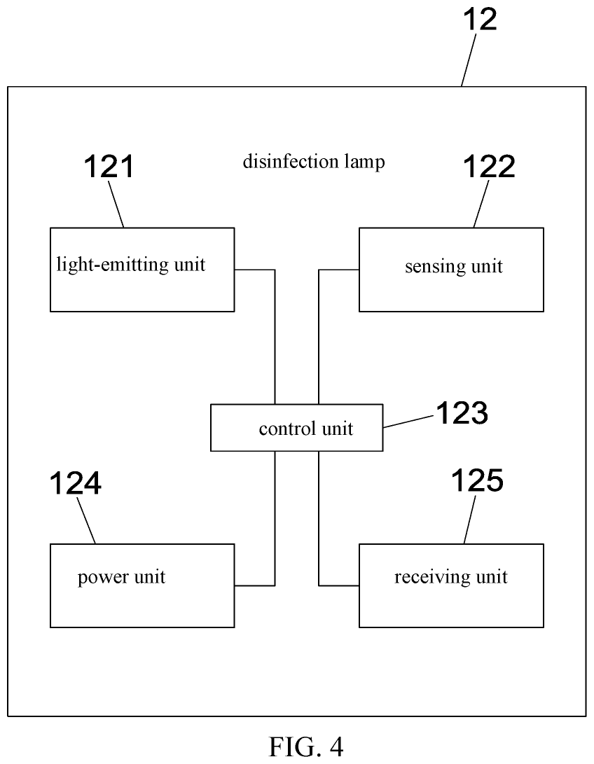

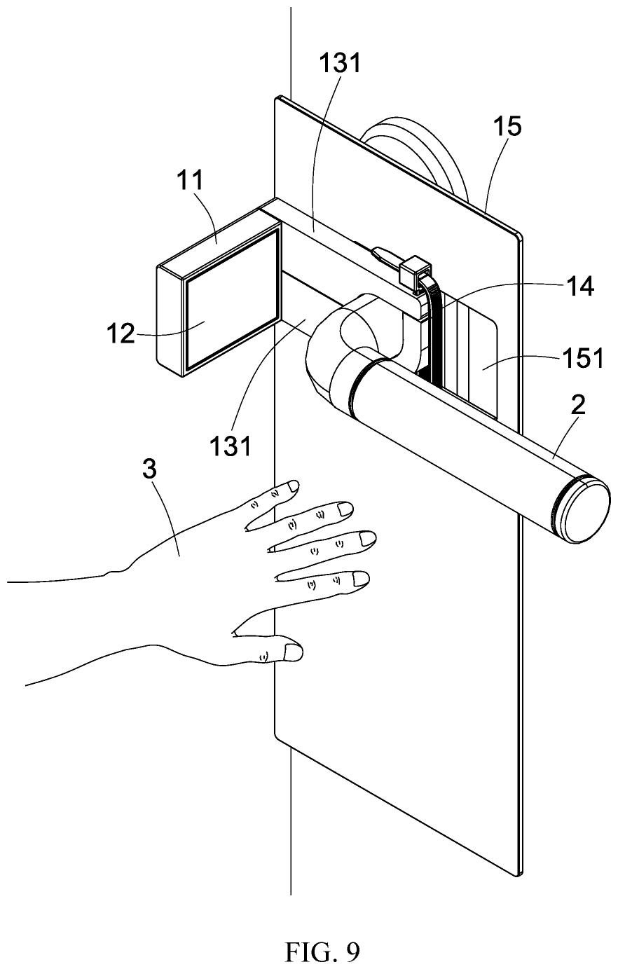

Referring to FIG. 4, the disinfection lamp 12 is configured on the main body 11 and includes a light-emitting unit 121, sensing unit 122, control unit 123, power unit 124, and receiving unit 125. The disinfection lamp 12 is used to emit light for disinfection, and the light may for example be ultraviolet light. The light-emitting unit 121 serves as the light source of the disinfection lamp 12 to generate light, and may for example be a UV-B ultraviolet lamp, UVC ultraviolet lamp, mercury lamp, metal halide lamp or xenon lamp, but the present is not so limited. The sensing unit 122 is used to sense a human body 3 (shown in FIG. 9), and may for example be an infrared sensing unit, light sensing unit, ultrasonic sensing unit or thermal sensing unit. The control unit 123 is used to control the light-emitting unit 121, and the power unit 124 provides electric power. The power unit 124 may for example be a battery and may be configured on the power receiving slot 111 of the main body 11 (shown in FIG. 3). The receiving unit 25 is used to receive a wireless signal, and may for example a wireless signal receiver, allowing the control unit 123 to control the light-emitting unit 121 to generate light according to the wireless signal. For example, a smart phone or remote controller may be used to emit the wireless signal to the receiving unit 125.

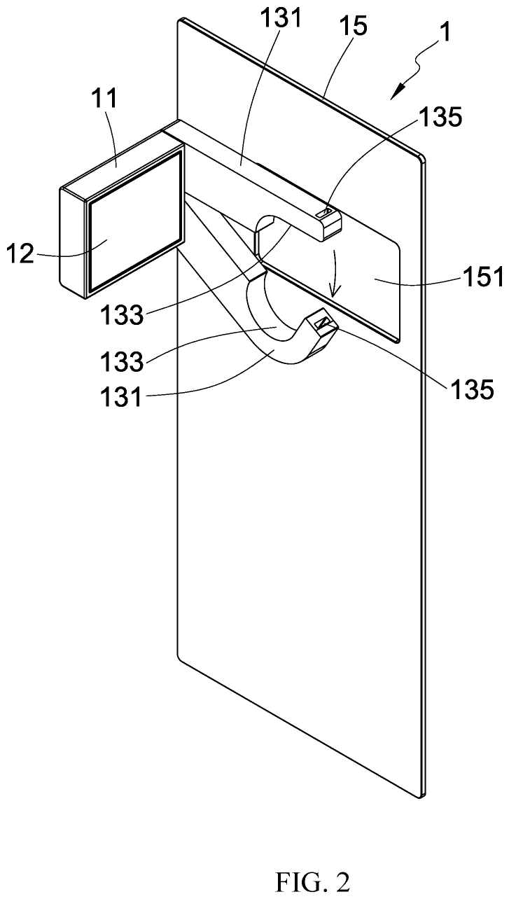

Referring to FIGS. 2 and 3, the clamping part 13 is configured on one side of the main body 1, and includes two clamping claws 131 opposite to each other, at least one of which is configured with a pivot shaft (shown in FIG. 3) to form an active opening or closing state of the two clamping claws 13 (shown in FIG. 2). Furthermore, each of the two clamping claws 131 is configured with a recess 133, allowing the two clamping claws to abut against each other to form a clamping space 134 when closed, and the two clamping claws 131 respectively have a through hole 135.

Referring to FIG. 7, the fixing part 14 is used to pass through the through holes 135 of the two clamping claws 131 to fix the two clamping claws 131 abutting against each other when they are closed.

The notice board 15 is configured on one side of the clamping part 13 and configured with an accommodating hole 151. The notice board 15 may be provided with signs such as trademarks, product descriptions, use of warnings or decorative patterns, etc., where the two clamping claws 131 of the clamping part 13 are located oppositely to one side of the accommodating hole 151 of the notice board 15.

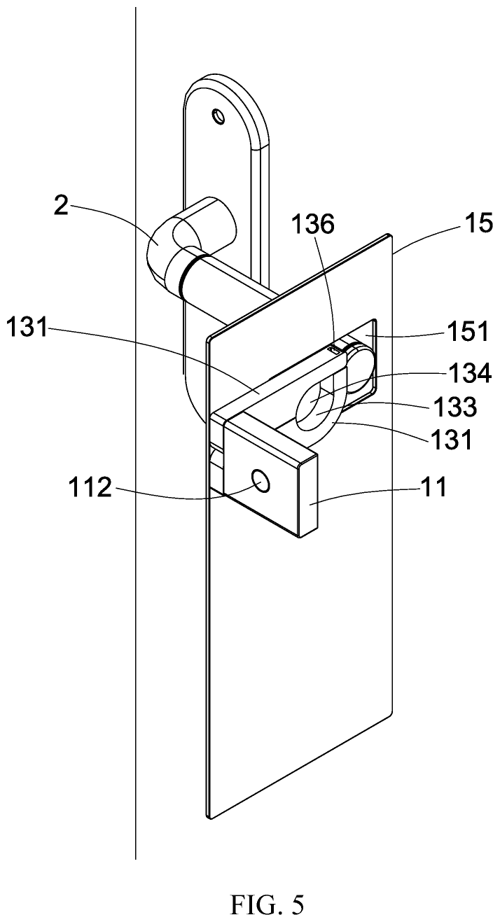

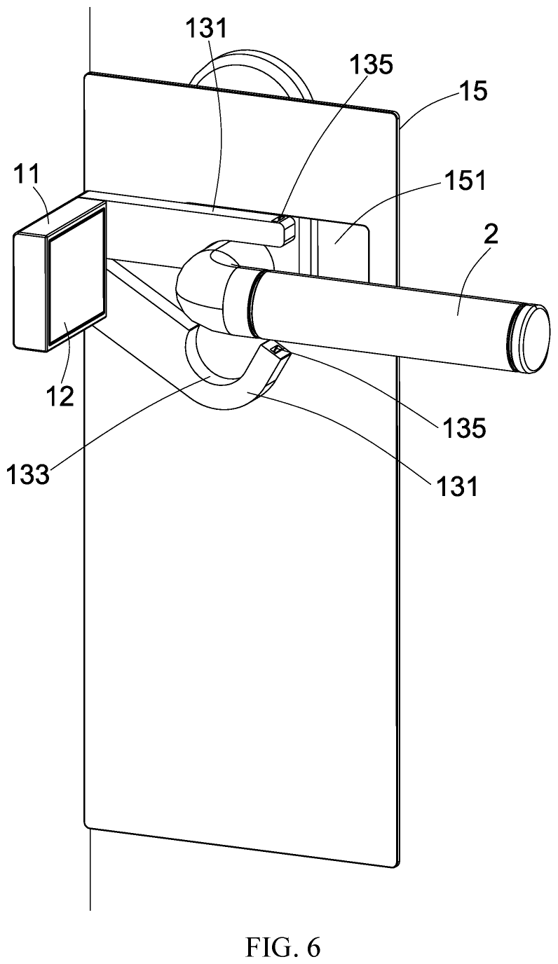

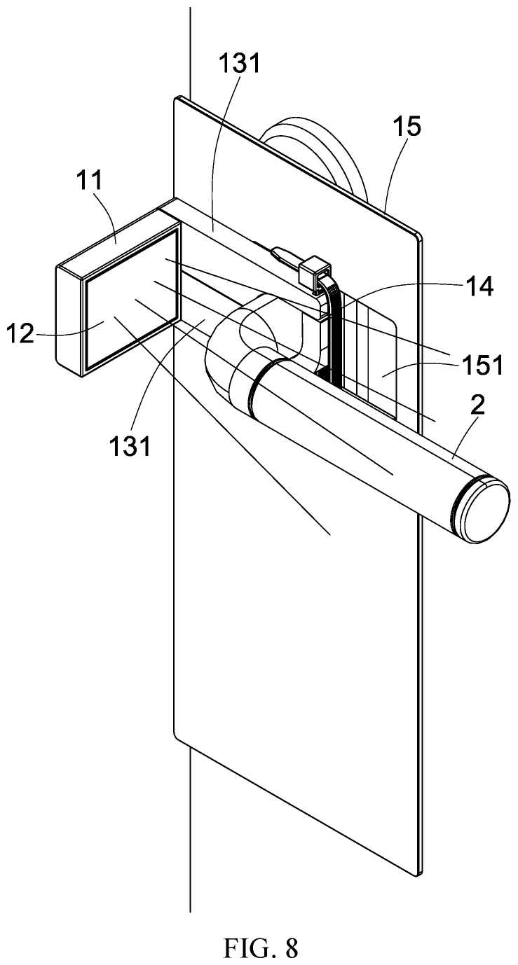

Referring to FIGS. 5 and 6, taking a door handle 2 as an example, the main body 11 is put around the door handle 2 through the accommodating hole 151 of the notice board 15, the position of the notice board 15 relative to the door handle 2 is adjusted, and the two clamping claws 131 are used to clamp the door handle 2 with the active opening and closing thereof next, referring to FIGS. 1 and 7, the fixing portion 14 is further passed through the through holes 135 of the two clamping claws 131, thereby fixing the two clamping claws 131 abut against each other together. Referring to FIG. 8, the disinfection lamp 12 can then emit light to illuminate the door handle 2 for disinfection. Referring to FIGS. 4 and 9, the sensing unit 122 is further used to detect around the door handle 2. When a human body 3 is sensed approaching. The sensing unit 122 notify the control unit 123. Thus, the control unit 123 controls the disinfection lamp 12 to stop emitting light, thereby avoiding harming the human body 3.

To sum up, the present invention can be quickly and simply installed on the door handle 2, and has a small volume without occupying space compared to conventional techniques. When the present invention is to be detached from the door handle, only the clamping claws 131 of the clamping part 13 abut against each other need to be opened after the fixing part 14 is removed. The fixing part 14 may for example be a fastening belt made of plastics, such that a sharp object such as scissors can then be used to remove the fixing part 14, being convenient and simple in operation. In addition, the ultraviolet sterilization has a better effect than the disinfectant sterilization.

I claim:

1. A clamping arm type disinfection device, comprising:
a main body;
a disinfection lamp, configured on said main body, and comprising a light-emitting unit, sensing unit, control unit, and power unit;
a clamping part, configured on one side of said main body, and comprising two clamping claws configured oppositely to each other, at least one of said two clamping claws configured with a pivot shaft, said two clamping claws abutting against each other to form a clamping space, and said two clamping claws respectively configured with a through hole;
a fixing part, passed through said through holes of said two clamping claws; and
a notice board, configured on one side of said clamping part, and configured with an accommodating hole.

2. The device according to claim 1, wherein said two clamping claws are respectively configured with a recess, allowing said two clamping claws to abut against each other to form said clamping space when closed.

3. The device according to claim 1, where said main body is configured with a power receiving slot.

4. The device according to claim 1, where said disinfection lamp comprises a receiving unit, and said receiving unit is a wireless signal receiver.

5. The device according to claim 1, wherein said two clamping claws of said clamping part are located at one side of said accommodating hole of said notice board.
